(19) United States
(12) Patent Application Publication
(10) Pub. No.: US 2010/0256233 A1
(43) Pub. Date: Oct. 7, 2010

(54) SYSTEMS, METHODS AND COMPOSITIONS FOR REDUCTION OF INJURY FROM RADIATION EXPOSURE

(76) Inventors: Jae Ho Kim, West Bloomfield, MI (US); Andrew Kolozsvary, Macomb, MI (US); Stephen L. Brown, Lasalle (CA); Jianguo Liu, Ann Arbor, MI (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 12/668,326

(22) PCT Filed: Jul. 10, 2008
(86) PCT No.: PCT/US08/69656

§ 371 Date: May 21, 2010

Related U.S. Application Data
(60) Provisional application No. 60/948,892, filed on Jul. 10, 2007.

(51) Int. Cl. A61K 31/19; A61K 31/16; A61P 17/16

(52) U.S. Cl. 514/557; 514/575

(57) ABSTRACT

Some embodiments of the invention comprise systems, methods and/or compositions for the systemic use of histone deacetylase inhibitors (“HDACi”) to protect against or alleviate the effects of radiation exposure, whether due to hostile actions, such as terrorist activity; through therapeutic radiation exposure; or through other methods of intentional or unintentional exposure to radiation, including but not limited to, total body irradiation.

### RELATED APPLICATIONS

This application claims priority to U.S. Provisional Patent Application No. 60/948,892 filed Jul. 10, 2007, which is hereby incorporated by reference as though fully set forth herein.

### GRANTS

Some work underlying the invention was supported by a Center for Medical Countermeasures against Radiation Injury (“CMCR”) grant from NIH/NIAID U19AI1067734-020005. The government may have certain rights in the invention.

### FIELD OF THE INVENTION

The invention relates generally to the field of radiation biology.

### BACKGROUND

Humans may be exposed to radiation from a variety of different sources. As some examples only, patients undergoing treatment for cancer might be exposed to radiation and can experience undesirable effects from irradiation of normal tissue. Cancer therapy by radiation is limited by the dose that normal tissue can tolerate. One approach is to target the radiation field precisely to the target volume. However, this approach is limited by cancers that can infiltrate adjacent normal tissues and the problems posed by normal skin and fat that overlie the diseased tissue. Moreover, in certain industrial or research settings, workers might be exposed to radiation, as some examples only, in the use or handling of radioactive materials as fuel or research materials. In addition, military personnel or civilians may be exposed to radiation as a result of military activities or attacks using nuclear materials, as one example only, as a result of a “dirty” nuclear bomb used as part of a terrorist attack.

The development of an effective mitigator of radiation injury before or following exposure to radiation, including without limitation, a total body irradiation (“TBI”) exposure, has the potential to benefit such persons. However, there are no currently known pharmacological approaches that mitigate such injury or lethality after a person is exposed to TBI. Although granulocyte colony-stimulating factor (“G-CSF”) is under investigation, and bone marrow transplants (“BMT”) have been used, their utility remains undetermined and certain risks arising from their use might outweigh potential benefits. For example, the risks of BMT may outweigh its benefits when the TBI dose is low because of associated toxicity.

Thus, a significant unmet need remains for improved systems, methods, and/or compositions to mitigate or protect against radiation exposures.

### BRIEF DESCRIPTION OF THE DRAWINGS

Certain embodiments of the present invention will now be described, by way of example, with reference to the accompanying drawings, in which:

FIG. 1 shows increases in acetylation of histone H3 relative to a control in the bone marrow of mice.

FIG. 2 shows that HDAC inhibition mitigates radiation injury to bone marrow cells.

FIG. 3 shows a comparison of groups of Balb/c mice exposed to TBI in the absence or presence of VPA at certain dose levels.

FIG. 4 shows a comparison of groups of Balb/c mice exposed to TBI in the absence or presence of VPA at additional dose levels.

FIG. 5 illustrates increased survival after TBI when an HDAC inhibitor was administered before and after TBI.

FIG. 6 shows kinetics of protection against and mitigation of death induced by TBI by an HDAC inhibitor.

Other aspects of the invention will be apparent to those skilled in the art after reviewing the drawings and the detailed description below.

### DETAILED DESCRIPTION

Without limiting the invention to only embodiments described herein and without disclaiming any other embodiments, some embodiments of the invention comprise systems, methods and/or compositions for the systemic use of histone deacetylase inhibitors (“HDACi”) to reduce, protect against, mitigate, alleviate, and/or ameliorate the effects of radiation exposure, whether due to hostile actions, such as terrorist activity; through therapeutic radiation exposure; or through other methods of intentional or unintentional exposure.

We investigated systems, methods, and compositions, including without limitation, pharmacological agents, in order to assess their potential to mitigate radiation injury. Even though HDACi have been shown to increase radiation-induced apoptosis in cancer cells, we tested whether HDACi might mitigate normal tissue radiation injury or decrease lethality following TBI. As part of our work, HDACi was administered to test animals in conjunction with TBI, and testing was conducted to determine the effects of the HDACi administration. We discovered that HDACi improve survival in mammals when administered either before or after TBI. As one example only, and without limitation, groups of mice were exposed to whole-body radiation doses between 5.5 and 7.0 Gy alone or in combination with an HDACi, such as valproic acid (“VPA”). We discovered an increase in survival whether VPA (400 or 600 mg/kg) was administered before or after TBI. Thus, we discovered that the systemic use of HDACi protects against or reduces injuries after TBI.

In accordance with some embodiments of the invention, without limitation, administration of an HDACi protects mammals from the lethal effects of whole body radiation exposure, minimizing normal tissue injury to bone marrow following an exposure to ionizing radiation and increasing the chance of survival following a whole body radiation exposure. It may be hypothesized that HDACi may increase survival after TBI based on reports demonstrating that HDACi stimulate the proliferation of bone marrow stem cells (1, 2). Indeed, some embodiments offer a new tool for bone marrow protection to radiation injury, where previously the only alternative was a bone marrow transplant, which is costly and risky.

As one example only, and without limitation, using the time for mice to lose 20% or more of their weight as the end point, we discovered that two HDACi, valproic acid and trichostatin-A, reduce lethality in a dose-dependent manner. HDACi were effective at reducing lethality when given either 24 hours before or 1 hour after TBI. The results indicate that HDACi can protect against and mitigate radiation-induced lethality.

HDACi such as those used in the nonlimiting examples described herein are FDA-approved and have been demonstrated to be safe for humans with minimal toxicities. HDACi have a documented safety record for systemic use in humans with a variety of neurodegenerative diseases and have recently received considerable interest for their direct anti-cancer effects and as a therapy to reduce graft versus host disease after an allogenic bone marrow transplant.

Systems, methods, and compositions comprising embodiments of the invention may be of benefit to, as some examples only, humans and other mammals exposed to radiation. As two nonlimiting examples, cancer patients exposed to radiation therapy could benefit from normal tissue radiation protection. Importantly, HDACi have been shown to be anti-cancer drugs when given alone and may potentiate the effect of radiation and cancer. Based on our results, HDACi appear to have the novel characteristic of simultaneously providing radiation protection/mitigation to normal tissue stem cells while causing tumors to be more sensitive to radiation. Yet cancer therapy by radiation is often limited by the dose that normal tissue can tolerate, and such therapy would be more efficacious if normal tissue complications did not exist or were mitigated. HDACi have the potential to reduce significantly normal tissue complications following radiation exposure. A second group of people that would benefit from HDAC inhibition therapy would be those accidentally exposed to radiation, including without limitation, military personnel or civilians exposed as a result of a terrorist attack by a “dirty” radiation bomb; victims of a radiological attack or nuclear disaster; or clean-up workers or other responders exposed after such events.

While an understanding of the precise mechanism by which HDACi may act is not necessary to practice embodiments of the invention, it would appear from complexities of the timing and sequence of treatment that the biological responses to HDACi depend on more than one mechanism.

Administration of nontoxic HDACi is known in several other disease conditions. HDACi have been developed for their anti-cancer activity because of their ability to regulate gene transcription involved with tumor cellular proliferation, differentiation and/or apoptosis. Some nonexclusive examples of HDACi include agents like valproic acid (Abbott Laboratories in US and Pfizer in UK), trichostatin, phenylbutyrate, depsipeptide (Fujisawa, Japan), SAHA (Aton Pharma, Tarrytown, N.Y.), and LAQ824 (Novartis Pharmaceuticals). These drugs are being studied in a variety of Phase I and II clinical trials, and have shown intriguing activity in the treatment of many kinds of cancer. HDACi have been reported to stimulate the proliferation of stem cells of the bone marrow (1, 2). Some researchers (3) observed that following a high-dose topical application there was a reduction in normal skin injury following radiation.

Histone hyperacetylation, a consequence of the action of HDACi, may lead to the upregulation of cell cycle inhibitors (p21, p27 and p16). Thus, HDACi may temporarily arrest bone marrow cells in the cell cycle and allow time for repair of radiation-induced DNA damage. The effects of HDACi after whole body radiation that increase the chance of surviving the exposure may be a result of histone hyperacetylation that leads to the upregulation of cell cycle inhibitors. However, embodiments of the invention are not limited to any particular mechanism of action and/or to only those disclosed herein.

An additional explanation might be that the reduction in lethality with administration of HDACi before irradiation is a result of increased repair of DNA damage. It has been shown in vitro that HDACi increase the rate of non-homologous end joining of DNA double-strand breaks (7). Thus, in vivo a time window of opportunity may exist for administration of HDACi to protect against a subsequent radiation exposure through a similar increase in DNA repair. The time window would depend on the bioavailability of the drug relative to the radiation exposure. After radiation exposure, our data are consistent with the mitigating effect of HDACi on radiation injury being mediated by the proliferative effect of HDACi on the proliferation of bone marrow cells.

It has been generally accepted since the early 1900s that stem cells are relatively sensitive to radiation-induced cytotoxicity. Indeed, even after low doses of radiation such as 0.5 Gy, a relatively large proportion of stem cells are killed. However, a normal response of stem cells is self-replication, and in response to a radiation, particularly a large dose, stem cells up-regulate proliferation and self-renewal.

The mechanism of action of HDACi is complex since HDAC inhibition affects numerous mechanisms including apoptosis, differentiation and transcription, etc. The role of HDACi in modulating immunity and inflammation has recently been elucidated (8). Wnt-1, a glycoprotein, has a role in many of these processes and is likely a key player in the differential effect of radiation on normal and many tumor cells. Wnt signaling has a major role in self-renewal of hematopoietic stem cells (9), and both VPA and TSA alter Wnt signaling in human and animal cells (7).

Our discovery shows that HDACi represent a new class of radiation protector and mitigator against total body irradiation capable of a significant reduction in injury even when administered after radiation exposure, as well as an agent to increase the chance of survival following a whole body radiation exposure.

Active compounds used to carry out embodiments of the present invention are histone deacetylase inhibitors. Examples of HDACi which may be used in accordance with the some embodiments of the present invention include, but are not limited to, Trichostatin A, Trichostatin C, butyric acid and butyric acid salts such as potassium butyrate, sodium butyrate, ammonium butyrate, lithium butyrate, phenylbutyrate, and sodium phenylbutyrate (“NaPBA”); stable butyrate derivatives, traponin, valproic acid, suberoylanilide hydroxamic acid (“SAHA”), and the like, as well as HDACi disclosed in publications such as U.S. Pat. Nos. 7,154,002 and 7,169,801, the disclosures of which are incorporated by reference in full.

The HDACi of some embodiments can be in the form of pharmaceutically acceptable salts. Such pharmaceutically acceptable salts may be used so long as they do not adversely affect the desired pharmacological effects of the compounds. The selection and production can be performed by a skilled artisan. Nonlimiting examples of pharmaceutically acceptable salts include alkali metal salts such as sodium salt or a potassium salt, alkaline earth metal salts such as calcium salt or a magnesium salt, salts with an organic base such as an ammonium salt, or a salt with an organic base such as a triethylamine salt or an ethanolamine salt.

It is expected that the timing and duration of HDACi treatment in humans will approximate those established for animal models. Similarly, the doses established for achieving results using such compounds in animal models, or for other clinical applications in humans, would be expected to be applicable in this context as well. Such factors may be adjusted according to principles and methods known to those of ordinary skill in the art.

In some embodiments without limitation, HDACi is administered and dosed in accordance with good medical practice, taking into account the clinical condition of the individual patient, the site and method of administration, scheduling of administration, patient age, sex, body weight and other factors known to medical practitioners. The “pharmaceutically effective amount” for purposes herein is thus determined by such considerations as are known in the art. The amount must be effective to achieve improvement of and/or protection against injury from radiation exposure, as measured or measurable according to criteria known to those of ordinary skill in the art.

HDACi can be administered in various ways. It can be administered alone or as an active ingredient in combination with pharmaceutically acceptable carriers, diluents, adjuvants and vehicles. HDACi can be administered orally, subcutaneously or parenterally including intravenous, intraarterial, intramuscular, intraperitoneal, and intranasal administration as well as intrathecal and infusion techniques, or by local administration or direct inoculation to the site of disease or pathological condition. Implants of the compounds are also useful. The patient being treated is a warm-blooded animal and, in particular, mammals including humans. The pharmaceutically acceptable carriers, diluents, adjuvants and vehicles as well as implant carriers generally refer to inert, non-toxic solid or liquid fillers, diluents or encapsulating material not reacting with the active ingredients of the invention. In some embodiments, doses that are effective at mice may be high compared to human doses, however, equivalent oral human doses needed for an effect are currently expected to be non-toxic.

It is noted that humans are treated generally longer than the experimental animals exemplified herein which treatment has a length proportional to the length of the disease process and drug effectiveness. The doses may be single doses or multiple doses over periods of time. The treatment generally has a length proportional to the length of the disease process and drug effectiveness and the patient species being treated. In some embodiments, HDACi have an effect when administered as a single dose after the radiation exposure (i.e. 1 hour).

When administering HDACi parenterally in some embodiments, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

When necessary, proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for the compositions of some embodiments. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the inhibitor(s).

Sterile injectable solutions can be prepared by incorporating the HDACi utilized in practicing some embodiments in the required amount of the appropriate solvent with various of the other ingredients, as desired.

A pharmacological formulation can be administered to the patient in an injectable formulation containing any compatible carrier, such as various vehicle, adjuvants, additives, and diluents; or HDACi utilized in some embodiments can be administered parenterally to the patient in the form of slow-release subcutaneous implants or targeted delivery systems such as monoclonal antibodies, vectored delivery, iontophoretic, polymer matrices, liposomes, and microspheres. Many other such implants, delivery systems, and modules are well known to those skilled in the art.

In some embodiments, without limitation, HDACi can be administered initially by intravenous injection to bring blood levels to a suitable level. The patient's levels are then maintained by an oral dosage form, although other forms of administration, dependent upon the patient's condition and as indicated above, can be used. The quantity to be administered and timing of administration may vary for the patient being treated.

### EXAMPLES

The following examples of some embodiments of the invention are provided without limiting the invention to only those embodiments described herein and without disclaiming any embodiments.

Methods and Materials

All studies were performed with prior approval and in accordance with institutional and national standards of animal care. The studies were approved by an Institutional Animal Care and Use Committee (“IACUC”) and were performed in an accredited AAALAC facility. Male BALB/c mice (Charles River Laboratories) 8 to 10 weeks old were allowed to acclimate for 1 week prior to the start of the study. Mice were maintained in environment-controlled (temperature and lighting) animal facilities, provided food and water ad libitum, and randomly assigned to experimental groups.

Histone Deacetylase (HDAC) Inhibitors

Two HDAC inhibitors were studied, valproic acid (“VPA”), a class I HDAC inhibitor, and trichostatin-A (“TSA”), a non-selective HDAC inhibitor. VPA (Depakene, Abbott Laboratories, Abbott Park, Ill.) was obtained from a hospital pharmacy. TSA was purchased from Sigma-Aldrich (St. Louis, Mo.).

Total-Body Irradiation (“TBI”)

TBI was delivered using a 185 TBq (5000 Ci) ¹³⁷Cs source (Mark I, J. L. Shepherd and Associates, San Fernando, Calif.). Doses ranged from sublethal exposures of 5.5 Gy to lethal exposures of 6.5 Gy and a supralethal exposure of 7.0 Gy. The dose rate was approximately 1 Gy per minute.

HDACi Activity

Western blot analyses were used to confirm the activity of VPA and TSA. Histone H3 and H4 acetylation activity of fresh mouse spleen and bone marrow after administration of VPA or TSA was measured by Western blot analyses ex vivo. Mice were administered two doses of HDAC inhibitors subcutaneously, one dose at 0 h (TSA, 0.5 mg/kg; VPA, 600 mg/kg) and one dose at 5 h (TSA, 0.5 mg/kg; VPA, 300 mg/kg). Blood marrow or spleens were harvested at 10 h.

Blood Counts

Twelve days after exposure to 5.5 Gy alone or in combination with either valproic acid (400 mg/kg administered i.p. 1 h after radiation exposure) or trichostatin A (0.5 mg/kg i.p. administered 1 h after radiation exposure), mice were anesthetized with ketamine (100 mg/kg) and xylazine (10 mg/kg) for blood collection. Blood (0.5 ml) obtained by cardiac puncture with a 25-gauge needle was placed into heparinized anticoagulant tubes and shipped to ANTECH Diagnostics (Alsip, Ill.) for analysis of red and white blood cell counts using their CBC differential small mammalian protocol. The same mice were used for the spleen colony-forming assay.

Spleen Colony-Forming Assay

The numbers of spleen colony-forming units were measured to assess the in vivo effect of HDACi on bone marrow cell survival. Groups of BALB/c mice were exposed to 5.5 Gy alone or in combination with HDACi as described. Spleens were excised and examined for colony formation 12 days after the sublethal TBI using a dissecting microscope.

Lethality

Survival was measured in mice after TBI with and without HDACi. Groups of mice were exposed to 6.5 or 7.0 Gy alone or in combination with HDACi, as described above. Survival was defined as the time from irradiation to the time of euthanasia, which occurred when animals had lost 20% or more of their body weight.

Results

HDACi Activity in Bone Marrow of Mice

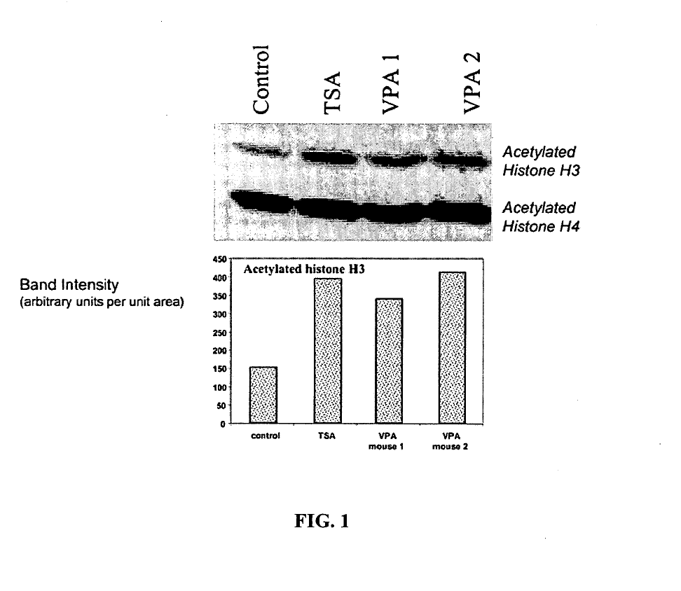

VPA is a class I HDAC inhibitor and is expected to increase acetylation of histone H3. TSA is not selective and is expected to increase acetylation of histones non-specifically. FIG. 1 is a graph showing the acetylation of histone H3 relative to a beta-actin control in test samples. As shown in FIG. 1, Western blot analyses for acetylated histones in bone marrow done 10 hours after the first dose (TSA: 0.5 mg/kg; VPA: 600 mg/kg) and 5 hours after the second dose (TSA: 0.5 mg/kg; VPA: 300 mg/kg) of HDACi showed a three to four-fold increase in acetylated form of histone H3 relative to the beta-actin control. Bars indicate individual mice. These studies established that both TSA and VPA acetylated histones in our model at the doses used.

VPA Increases Endogenous Spleen Colony Formation

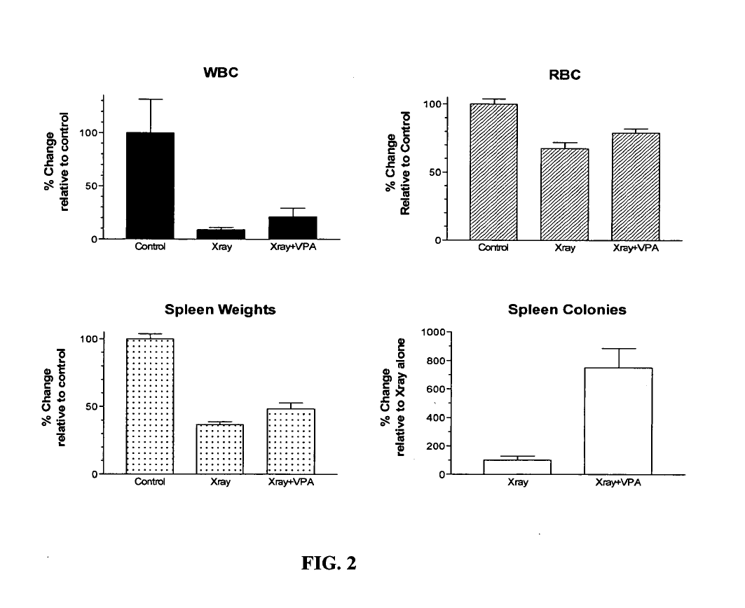

The effect of HDAC inhibition on the survival of bone marrow stem cells was assayed using a variant of the spleen colony assay of Till and McCulloch in which endogenous nodules on the spleens of mice are counted after a sublethal dose of TBI (4-6). FIG. 2 illustrates the results. Twelve days after sublethal irradiation (5.5 Gy) plus HDAC inhibitor (400 mg/kg valproic acid given i.p. 1 h after X rays), the number of spleen colonies was sevenfold higher (P<0.001) than in mice exposed to 5.5 Gy alone (lower right panel). Leukopenia (upper left panel) and erythropenia (upper right panel) were significantly improved in groups of mice receiving HDACi in combination with radiation compared to that in mice receiving radiation alone. Also, spleen weights in mice that were reduced after radiation alone exposure were significantly increased (p<0.05) in mice receiving HDACi plus radiation (lower left panel). These results are consistent in showing that HDACi mitigate radiation injury to the bone marrow.

Effect of HDACi Dose on Lethality of TBI

The effect of different doses of HDACi on the survival of irradiated mice was studied.

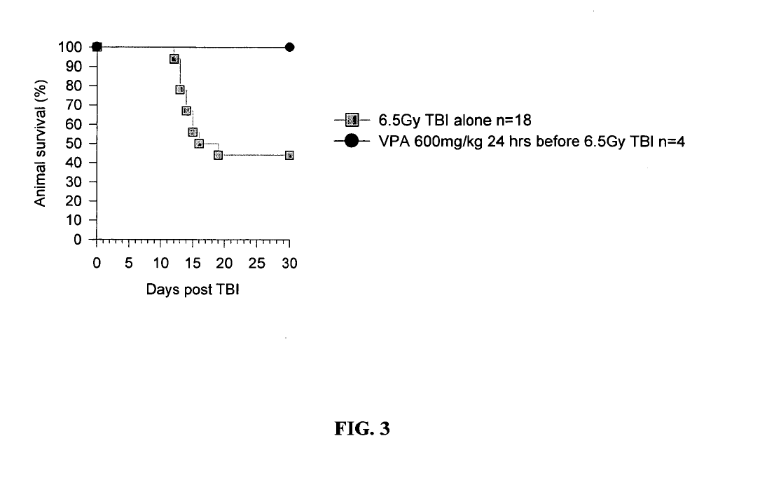

Our data show that HDACi protects mice from TBI. FIG. 3 shows a comparison of groups of Balb/c mice exposed to total body irradiation in the absence or presence of VPA. Half the mice were dead by day 16 following 6.5 Gy whereas no mice died when VPA preceded radiation exposure by one day.

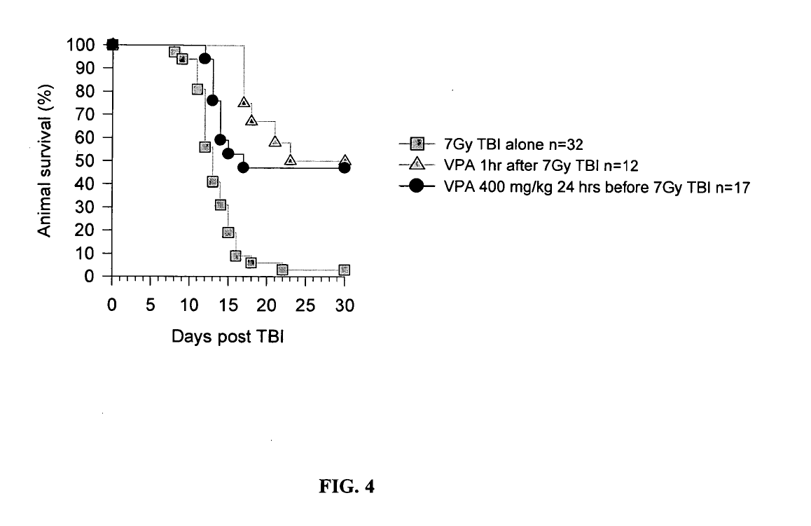

Our data also show that HDACi enhances survival when given after TBI. FIG. 4 shows a comparison of groups of Balb/c mice exposed to total body irradiation in the absence or presence of VPA. The time to 50% lethality increased from day 13 for 7 Gy TBI alone (7 Gy) to day 17 when VPA was given 1 day before TBI and to day 23 when VPA followed radiation by 1 hour.

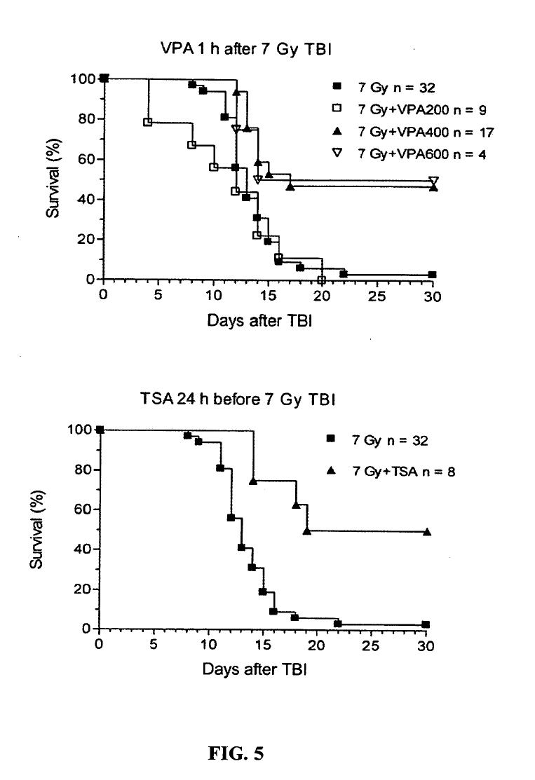

VPA given 24 h before radiation increased the chance that mice would survive a lethal dose TBI. FIG. 5 shows a comparison of groups of Balb/c mice exposed to total body irradiation in the absence or presence of VPA. Groups of BALB/c mice were exposed to TBI alone (7 Gy) alone or in combination with VPA at 200, 400 or 600 mg/kg. When VPA followed TBI by 1 h, a dose of at least 400 mg/kg resulted in increased survival. VPA at 200 mg/kg was not effective (P <0.05 compared with VPA at 400 mg/kg, log rank test). FIG. 5 illustrates the increased survival after 7 Gy TBI when TSA (0.5 mg/kg) was administered 24 h before TBI. Without VPA, no mice survived a radiation dose of 7 Gy, and half of the mice had died by 12 days after irradiation. A single dose of 200 mg/kg VPA yielded no benefit. A dose of 400 or 600 mg/kg extended the median survival to 14 to 17 days respectively, resulted in about half of the mice surviving for at least 2 months.

Time and Sequence of Treatment with HDACi on Lethality of TBI

The effect of HDACi on the survival of mice after a radiation exposure was studied for various treatment sequences and times. For both the lethal (6.5 Gy), and the supralethal (7 Gy) radiation dose, VPA administration enhanced the survival of irradiated mice. Less than half of a group of 18 mice survived 6.5 Gy TBI alone, whereas VPA (600 mg/kg) administered 24 h before 6.5 Gy resulted in 100% survival (n=4). Similarly, 50% of a group of 32 mice exposed to 7 Gy were dead 13 days after exposure (i.e. ED₅₀=13 days), whereas VPA (400 mg/kg) administered 24 h before or 1 h after the radiation exposure resulted in an improved ED₅₀ of 17 days (n=17) and 26.5 days (n=12), respectively. All mice that survived until day 17 or day 18 after radiation exposure appeared to be healthy and lived until the end of the study (at least 30 days).

Significant radiation protection was observed when VPA was given 24 h before or 1 h after whole-body irradiation (FIGS. 3-5). VPA (600 mg/kg) given 24 h before 6.5 Gy TBI increased survival significantly (one-sided logrank test, P<0.05). For 7.0 Gy TBI, 600 mg/kg VPA given 24 h before or 1 h after irradiation increased survival significantly (P<0.0001). However, it is of note that although HDACi reduced the lethality of TBI when administered 24 h prior to irradiation and between 1 and 6 h after exposure, when VPA was given 1 h before radiation exposure, survival was not increased (data not shown).

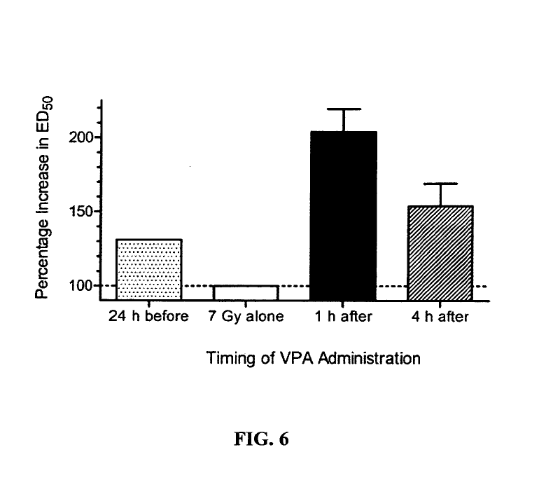

FIG. 6 summarizes the effect of the timing and sequence of administration of VPA with respect to radiation exposure. That figure shows kinetics of protection against and mitigation of death induced by TBI by an HDACi. The mean percentage increase in survival time in days is shown for groups of BALB/c mice receiving TBI and VPI at 400 mg/kg relative to survival time after TBI (7 Gy) alone. Error bars represent the range of survival times. n=17, 32, 12 and 16 mice per group for 24 h before, 7 Gy alone, 1 h after, and 4 h after, respectively.] FIG. 4 shows a comparison of groups of Balb/c mice were exposed to total body irradiation in the absence or presence of VPA. The time to 50% lethality increased from day 13 for 7 Gy TBI alone (7 Gy) to day 17 when VPA was given 1 day before TBI and to day 23 when VPA followed radiation by 1 hour. An increase in survival was observed when VPA was administered either 24 h before or shortly (1 h or 4 h) after the exposure.

Each of the references identified herein is hereby incorporated by reference as though fully set forth herein.

While the present invention has been particularly shown and described with reference to the foregoing preferred and alternative embodiments, it should be understood by those skilled in the art that various alternatives to the embodiments of the invention described herein may be employed in practicing the invention without departing from the spirit and scope of the invention as defined in the following claims. It is intended that the following claims define the scope of the invention and that the method and apparatus within the scope of these claims and their equivalents be covered thereby. This description of the invention should be understood to include all novel and non-obvious combinations of elements described herein, and claims may be presented in this or a later application to any novel and non-obvious combination of these elements. The foregoing embodiments are illustrative, and no single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements.

### REFERENCES

1. J. C. Young, S. Wu, G. Hansteen, C. Du, L. Sambucetti, S. Remiszewski, A. M. O'Farrell, B. Hill, C. Lavau, and L. J. Murray. Inhibitors of histone deacetylase promote hematopoietic stem cells renewal. Cytotherapy. 6:328-336. 2004).
2. L. De Felice, C. Tatarelli, M. G. Mascolo, C. Gregorj, F. Agostini, R. Fiorini, V. Gelmetti, S. Pascale, F. Padula, and C. Nervi. Histone deacetylase inhibitor valproic acid enhances the cytokine-induced expansion of human hematopoietic stem cells. Cancer Res. 65:1505-1513. 2005). A. J. Becker, E. A. McCulloch, and J. E. Till. Cytological demonstration of the clonal nature of spleen colonies derived from transplanted mouse marrow cells. Nature. 197:452-454. 1963).
3. Y. L. Chung, A-J. Wang, and L-F. Yao. Antitumor histone deacetylation inhibitors suppress cutaneous radiation syndromes. Implications for increasing therapeutic gain in cancer radiotherapy. Mol. Cancer Ther. 3:317-325. 2004).
4. E. A. McCulloch, and J. E. Till. The radiation sensitivity of normal mouse bone marrow cells, determined by quantitative marrow transplantation into irradiated mice. Radiat. Res. 13:115-125. 1960).
5. J. E. Till, and E. A. McCulloch. A direct measurement of the radiation sensitivity of normal mouse bone marrow cells. Radiat. Res. 14:213-222. 1961).
6. M. Wojewodzka, M. Kruszewski, I. Buraczewska, W. Xu, E. Massuda, J. Zhang, and I. Szumiel. Sirtuin inhibition increases the rate of non-homologous end-joining of DNA double strand breaks. Acta. Biochim. Pol. 54:63-69. 2007).
7. J. L. Brogdon, Y. Xu, S. J. Szabo, S. An, F. Buxton, D. Cohen, and Q. Huang. Histone deacetylase activities are required for innate immune cell control of Th1 but not Th2 effector cell function. Blood. 109:1123-1130. 2007).
8. T. Reya, A. W. Duncan, L. Ailles, J. Domen, D. C. Scherer, K. Willert, L. Hintz, R. Nusse, and I. L. Weissman. A role for Wnt signalling in self-renewal of haematopoietic stem cells. Nature. 423:409-414. 2003.

What is claimed is:

1. A method of mitigating injury to a mammal exposed, or at risk of exposure, to radiation, comprising the steps of:
providing a composition comprising at least one histone deacetylase inhibitor, and
administering systemically to the mammal a pharmaceutically effective amount of the composition.

2. The method of claim 1, in which the composition comprises a pharmaceutically acceptable carrier.

3. The method of claim 1 or claim 2, in which the step of administering comprises oral, subcutaneously, intravenous, intraarterial, intramuscular, intraperitoneal, intranasal, or intrathecal administration.

4. The method of claim 1, 2, or 3, in which the mammal is a human.
